(19) United States
(12) Patent Application Publication
(10) Pub. No.: US 2026/0024692 A1
(43) Pub. Date: Jan. 22, 2026

(54) COOLING DEVICE FOR STIMULATION UNIT OF ELECTROMAGNETIC STIMULATION APPARATUS

(71) Applicant: ADDON TECH LTD., TAIPEI CITY (TW)

(72) Inventors: SHIH-TU YEN, TAIPEI CITY (TW); YEN-LUNG LIN, TAIPEI CITY (TW)

(21) Appl. No.: 18/773,608

(22) Filed: Jul. 16, 2024

(51) Int. Cl. H01F 27/22; A61N 2/02

(52) U.S. Cl. CPC H01F 27/22; A61N 2/02

(57) ABSTRACT

In a cooling device for a stimulation unit of an electromagnetic stimulation apparatus, the stimulation unit includes a coil assembly, the cooling device includes a casing having an accommodating space for accommodating the coil assembly and a flexible bag, the casing includes a front cover which is a disk structure, the coil assembly is fixed to a side of the front cover by an encapsulant, the periphery of the front cover is provided with multiple first fixed parts; a rear cover, which is a cover structure having a bottom and a circular lateral portion, an edge of the circular lateral portion is fixed with multiple second fixed parts opposite the first fixed parts; and the flexible bag is filled with a coolant. After the rear cover and the front cover are installed, the flexible bag provides a cooling effect to the coil assembly through convection or conduction.

### BACKGROUND OF THE DISCLOSURE

### Technical Field

The present disclosure relates to the field of cooling, and more particularly relates to a cooling device for a stimulation unit of an electromagnetic stimulation apparatus, in which the stimulation unit of the electromagnetic stimulation apparatus can quickly replace a cooling source that is suitable for users to maintain a tolerable temperature during repeated stimulations for health improvements, and prevent the unit, device and apparatus from being damaged by collisions due to the separate placement of heat sources, since the expensive heating coils and cover are packaged as a whole.

### Description of the Related Art

The stimulation signal from the nerves can be regarded as an electrical signal, which is used to stimulate various parts of the body (e.g., organs, tissues, muscles, etc.), causing the corresponding parts of the body to produce a corresponding action or interference.

Existing devices that use alternating magnetic fields to stimulate electrical signals to affect various parts of the body are commonly referred to as electromagnetic stimulation devices, such as Transcranial Magnetic Stimulation (TMS). The devices of this sort are mainly used for studying neurological functions and treating mental illnesses. With reference to FIG. 1 for the block diagram of common system components, the components other than an external power supply 101 can be regarded as the components of the electromagnetic stimulation device. In practice, the external power supply 101 typically provides power to a voltage supply unit 102 under the specification of the mains power AC 110V or AC 220V. The voltage supply unit 102 is used to rectify and filter the AC power supplied by the external power supply 101 and further convert the power into the rated voltage and current provided for the normal operation of a control unit 103. In addition, the AC power supplied by the external power supply 101 is rectified, filtered and converted into the rated voltage and current of the electromagnetic stimulation device, which is provided for an energy storage unit 104 to store energy. The control unit 103 controls to turn on a current switch 105, conduct the energy storage unit 104 and a stimulation unit 106, discharge the stimulation unit 106, and generate a magnetic field and heat when stimulation is desired. The general structure of the stimulation unit 106 is generally a three-dimensional coil made of copper wire coils, whose function in the device is to generate a magnetic field through alternating currents and serve as a stimulation end of the electromagnetic stimulation device, i.e., the main generator of magnetic field and heat to affect the nearby organisms.

A related-art electromagnetic stimulation apparatus requires an extremely high voltage to generate high current variation, which in turn generate a large magnetic field at the coil used for stimulation, in order to effectively stimulate the brain or nerves. If the stimulation threshold is expected to be reached, the voltage must be at least 300 volts, and commonly used voltages often exceed 1,000 volts, depending on the depth of the stimulation target and neural conditions. In addition, there are restrictions on the operating frequency, the stimulation frequency range of the human body generally falls in the range of 0.5-50 Hz, and the stimulation must operate continuously for 10 min. or more. When the system is operating under these conditions, the coil will continue to generate heat, resulting in a continuous increase in temperature. At the same time, since the surface temperature of the coil normally exceeds the acceptable range of human body within 30 seconds and 100 degrees Celsius within 5 minutes, and since the magnetic field has a very short effective range and is usually applied close to the skin, this will cause the coil temperature to rise to a level that is beyond the ability of the subject to tolerate or produce an uncomfortable somatosensory temperature.

In this regard, there have been various cooling means to improve the aforementioned drawbacks, and they include: (1) Coil resistance reduction type: Taking into consideration that resistance directly affects heat generation, the resistance can be reduced to effectively delay temperature rise. However, this will also lead to decrease in the efficiency of the magnetic field, out of focus of the stimulation range, overweight and other problems. (2) Intensity or frequency reduction type: Using the principle of reducing current flux to reduce heat generation as a cooling means, but it will also directly reduce the stimulating effect. (3) Heat resistant type: A medium is added between the coil and the skin to reduce the thermal conduction speed. However, the distance between the coil and an organism directly affects the stimulating effect, so the coil must be close to the skin, and a usable heat-resistant material cannot be too thick, resulting in poor stimulating effect. (4) Replacement type: The overheated coil is directly removed and replaced with a new one. Since the coil is a high voltage, high current and high heat generating component, the installation protection is particularly high, and during the process, a professional unit is required to properly handle the disassembly and afford the price of a multiple of coils. (5) Air-cooled type: The heat is carried away by blowing air on the coil. However, because the temperature rises very quickly, it requires a large amount of airflow to be effective. Due to the interference of magnetic field, the fan cannot be installed directly near the coil and must be conducted from other places in order to cool the coil, resulting in a very large wind cut and motor sound and indirectly leading to discomfort to the user. (6) Liquid circulation cooling type: The coolant flows through the coil to carry away the heat, and it surely can solve the problem of overheating the coil. However, the disadvantage is that immersing the conductive coils in the liquid increases the risk and maintenance, as well as the size, weight, and cost of the entire system.

In view of the fact that when an electromagnetic stimulation apparatus uses a direct current to perform periodic charge-stop cycles, the temperature of the stimulation unit (i.e., the coil temperature) will gradually rise, leading to the problem that the electromagnetic stimulation apparatus is not suitable and can no longer be used for the nearby organisms, and it is well known that all cooling means such as raising the substrate, lowering the efficiency, adopting the heat resistance of a dielectric material, changing the heat source, air cooling, liquid cooling, etc. have the same problems. At present, there is no better solution except tolerating the shortcomings of liquid cooling. Up to now, medical institutions that do not purchase high-end liquid-cooled apparatus are constantly faced with this difficult problem. If a dedicated air cooler is used, the patient will have to deal with a noise level that is louder than a vacuum cleaner, affecting the quality of medical care. In addition, for the sake of patients, some doctors use the coil replacement method. However, since the electromagnetic stimulation apparatus is a high-voltage device, the replacement procedure is complicated and may cause interruptions in treatment, and it is more likely to damage or burn the connectors due to poor contact during the process. Since the coils are expensive, medical institutions cannot keep too many replacement coils on hand at all times. As a result, doctors today have to prepare ice buckets to cool down the coils at any time during treatment. Furthermore, the maintenance and price of liquid cooling are also the main reasons why electromagnetic stimulation apparatus cannot be commonly used at home. Therefore, a simple and cheap coil cooling method is actually required to improve the current medical situation and popularize the electromagnetic stimulation apparatus, and the team of the present disclosure conceived and proposed a cooling device for the stimulation unit of the electromagnetic stimulation apparatus to improve various deficiencies derived from conventional methods and allow users to repeatedly perform electromagnetic stimulation on the surface of human body.

### SUMMARY OF THE DISCLOSURE

It is a primary objective of the present disclosure to overcome the problems of the related art by providing a cooling device for a stimulation unit of an electromagnetic stimulation apparatus. That is to say, for the coil assembly as a stimulation unit, after a short period of time of repeated stimulation actions, it can still maintain the temperature acceptable by the user's body and facilitate the implementation, and for the heat source and the cover with a modular design, the corresponding parts or components replaced can be quickly and safely.

To achieve the aforementioned objective, the present disclosure discloses a cooling device for a stimulation unit of an electromagnetic stimulation apparatus, the stimulation unit includes a coil assembly having an accommodating space that contains a casing, and an opening formed on the coil assembly, the accommodating space is provided for accommodating the coil assembly, the opening is provided for passing a plurality of power wires electrically connected to the coil assembly. The casing includes: a front cover which is a disk structure, and the coil assembly is fixed to a side of the front cover, where the periphery of the front cover is provided with a plurality of first fixed parts, and the front cover and the coil assembly are fixed as a whole by an encapsulant; and a rear cover which is a cover structure having a bottom and a circular lateral portion, an edge of the circular lateral portion is provided with a plurality of second fixed parts opposite the first fixed parts, the first fixed part are fixed and configured to be corresponsive to the second fixed part, and the accommodating space is defined between the front cover and the rear cover; and a flexible bag is installed in the accommodating space and filled with a coolant; wherein, after the rear cover and the front cover are installed, the flexible bag provides a cooling effect to the coil assembly through convection or conduction.

Preferably, the rear cover and the flexible bag are fixed as a whole.

Preferably, the bottom of the rear cover is provided with a pushing portion, the pushing portion has a ring edge and a pushing member embedded in the ring edge and pivoted with the ring edge, and when the pushing member rotates clockwise or counterclockwise rotation under an external force, the pushing member moves close to or away from the front cover to change the size of the accommodating space, so as to move all or part of the flexible bag accordingly to adjust the cooling performance of the flexible bag on the coil assembly.

Preferably, the first fixed part and the second fixed part are magnetic attachment structures.

Preferably, the coolant is water, or a non-metallic liquid with a magnetic permeability smaller than 1*10\hat{ }−5 Henrys/meter (H/m) and a specific heat capacity greater than 1,000 Joules/(kg*K).

Preferably, the flexible bag is directly in contact with the coil assembly; or indirectly in contact with the coil assembly with a gap smaller than 1 cm.

In summation of the description above, the present disclosure provides a cooling device for a stimulation unit of an electromagnetic stimulation apparatus, in which the front cover and the rear cover are combined in an easy-to-disassemble mechanism, and the heat source and front cover are modularized for easy replacement, so that the coil assembly that generates the corresponding heat source during the operation of the magnetic stimulation can be quickly wrapped inside, and the distance and state of the flexible bag with cooling effect and the coil assembly can be adjusted through the pushing member at the special position of the rear cover by means of a rotary movement, thus achieving the quick heat dissipation effect.

### BRIEF DESCRIPTION OF THE DRAWINGS

FIG. 1 is a schematic block diagram showing the cooling components of a cooling device of the present disclosure;

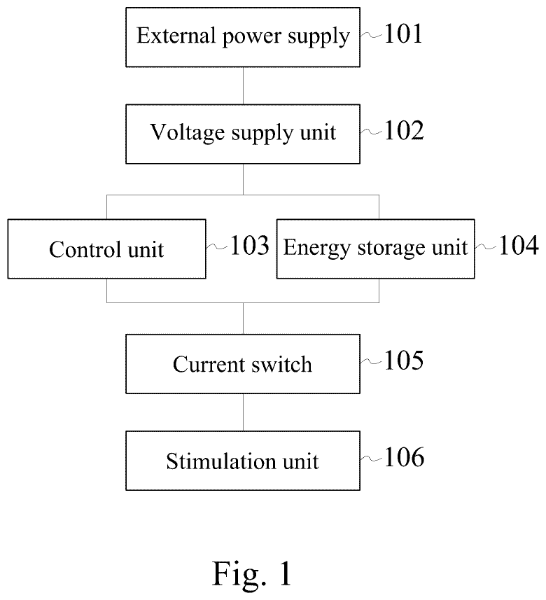

FIG. 2 is a perspective view of a cooling device of the present disclosure;

FIG. 3 is a disassembled view of a cooling device of the present disclosure;

FIG. 4 is a schematic view of a coil assembly fixed to a front cover of the present disclosure;

FIG. 5 is a schematic view of a flexible bag placed in a rear cover of the present disclosure;

FIG. 6 is a schematic view showings the front and back of a cooling device of the present disclosure;

FIG. 7 is a schematic view of withdrawing a pushing member of the present disclosure; and

FIG. 8 is a schematic view of pushing in a pushing member of the present disclosure.

### DETAILED DESCRIPTION OF THE PREFERRED EMBODIMENTS

In order to enable those having ordinary skill in the art to clearly understand the contents of the present disclosure, the following descriptions and drawings are provided for reference.

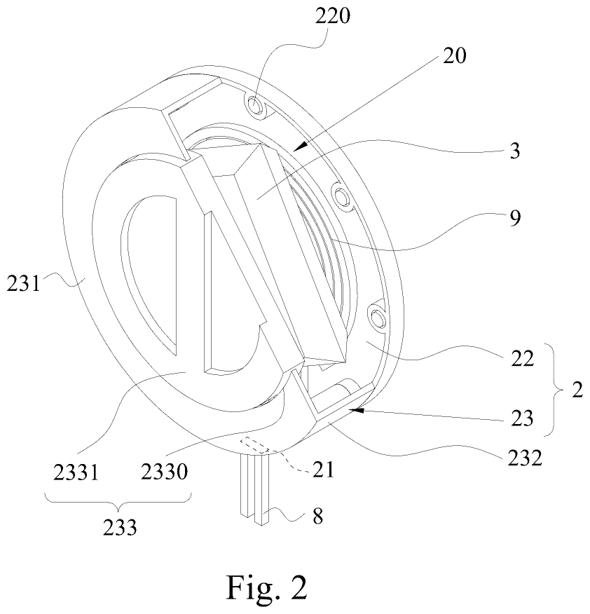

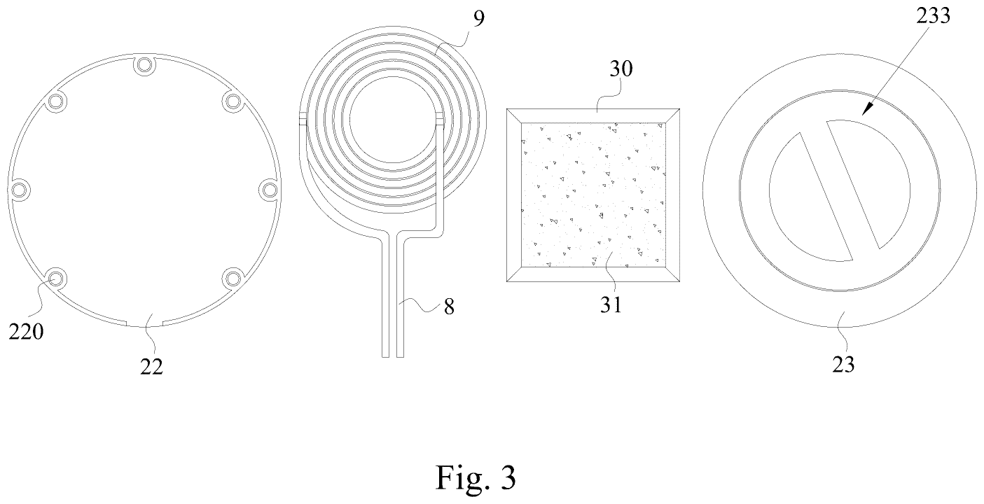

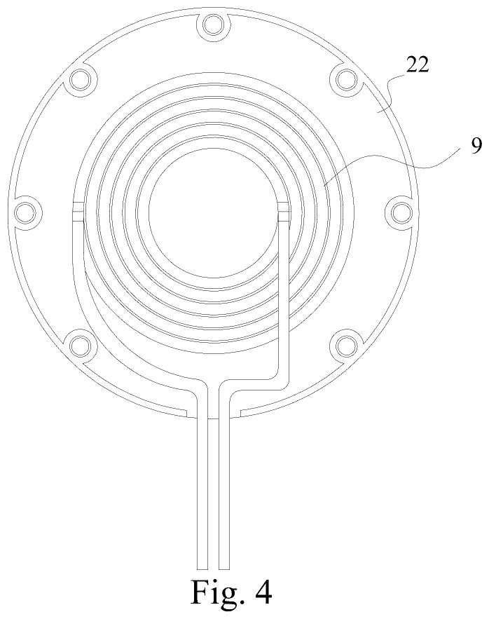

With reference to FIGS. 2 to 8 for the perspective view and disassembled view of a preferred embodiment of the present disclosure, the schematic view of a coil assembly fixed to a front cover, the schematic view of a flexible bag placed in a rear cover, and the schematic view showing the front and back of a cooling device, and the schematic views of withdrawing and pushing in a pushing member of the present disclosure respectively, the present disclosure discloses a cooling device for a stimulation unit of an electromagnetic stimulation apparatus, and the stimulation unit is a coil assembly 9, for example, it is wound with copper wire or aluminum wire and can be O-shaped, 8-shaped, tri-glide-slide-shaped, H-shaped, spiral-shaped, swirl-shaped, butterfly-shaped, spring-shaped or clover-shaped. The cooling device includes a casing 2 and a flexible bag 3. The casing 2 has an accommodating space 20 inside and an opening 21 formed on the casing 2. The accommodating space 20 is provided for accommodating the coil assembly 9 and the flexible bag 3, and the flexible bag 3 is made of a polyethylene material and filled with a coolant 31, wherein the coolant 31 can be water or a non-metallic liquid with a magnetic permeability smaller than 1*10\hat{ }−5 Henrys/meter (H/m) and a specific heat capacity greater than 1,000 joules/(kg*K), and the opening 21 is provided for passing a plurality of power wires 8 electrically connected to the coil assembly 9. Further, the casing 2 includes a front cover 22 and a rear cover 23 which are assembled easily. The front cover 22 is a disk structure, and the coil assembly 9 is fixed to a side of the front cover 22. Further, the front cover 22 and the coil assembly 9 are fixed as a whole by an encapsulant as shown in FIG. 4. Therefore, the front cover 22 and the coil assembly 9 are combined to form a heat source module, and this concept can prevent the coil assembly 9 from being damaged by collisions caused by replacing the coil assembly 9 alone, especially when the appearance of the coil assembly 9 makes it difficult for its replacement. After the rear cover 23 and the front cover 22 are assembled, the flexible bag 3 provides a cooling effect to the coil assembly 9 by convection or conduction. To improve the convenience of replacing the cooling source, the rear cover 23 and the flexible bag 3 are fixed as a whole. In other words, when the rear cover 23 is removed, the flexible bag 3 will be removed simultaneously.

To improve the convenience of the disassembling, the periphery of the front cover 22 is provided with a plurality of first fixed parts 220, the rear cover 23 is a cover structure having a bottom 231 and a circular lateral portion 232, and an edge of the circular lateral portion 232 is provided with a plurality of second fixed parts 230 corresponding to the first fixed parts 220 respectively, such that the quantity and position of the first fixed parts 220 correspond to those of the second fixed parts 230, and the first fixed parts 220 and the second fixed parts 230 are fixed to each other, and the accommodating space 20 is formed between the front cover 22 and the rear cover 23. In other words, the first fixed part 220 and the second fixed part 230 are configured to be corresponsive to each other and provided for combining and separating the front cover 22 and the rear cover 23 quickly. For example, the first fixed part 220 and the second fixed part 230 are magnetic attachment structures or tenon structures which allow the front cover 22 and the rear cover 23 to be assembled and disassembled conveniently and quickly, so that when the coolant 31 inside the flexible bag 3 obviously loses its cooling effect, the casing 2 can be removed conveniently for a quick replacement. Therefore, after the rear cover 23 and the front cover 22 are assembled, the flexible bag 3 can provide a cooling effect to the coil assembly 9 by convection or conduction.

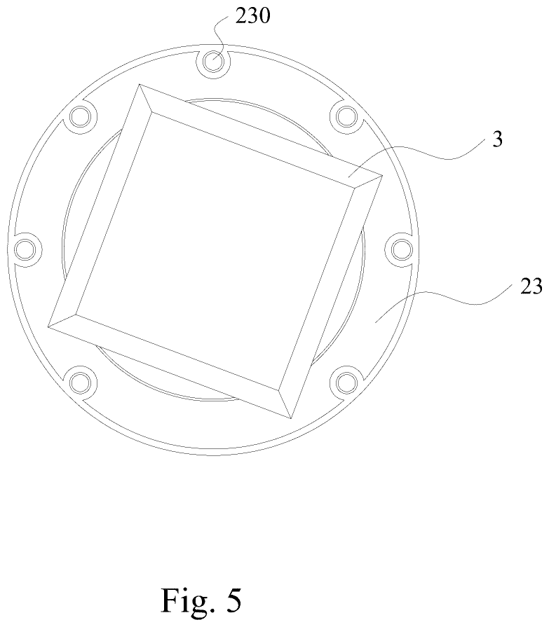

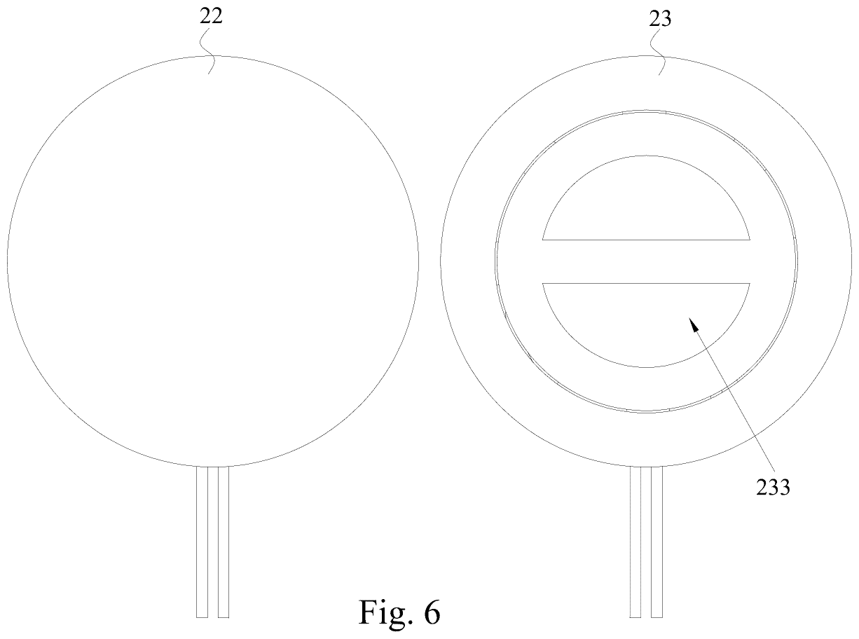

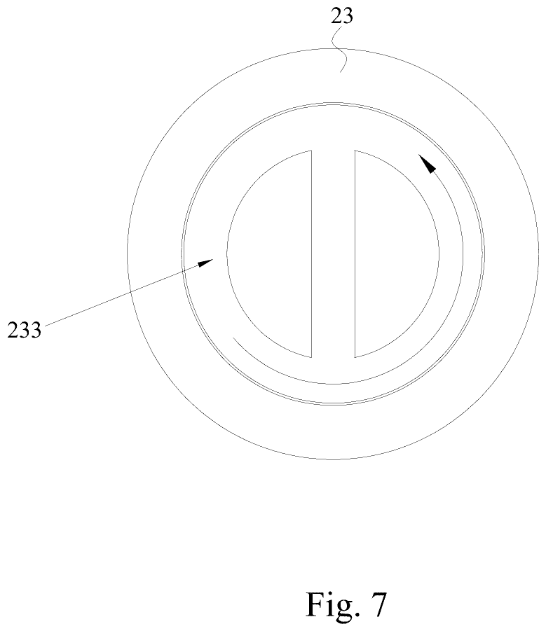

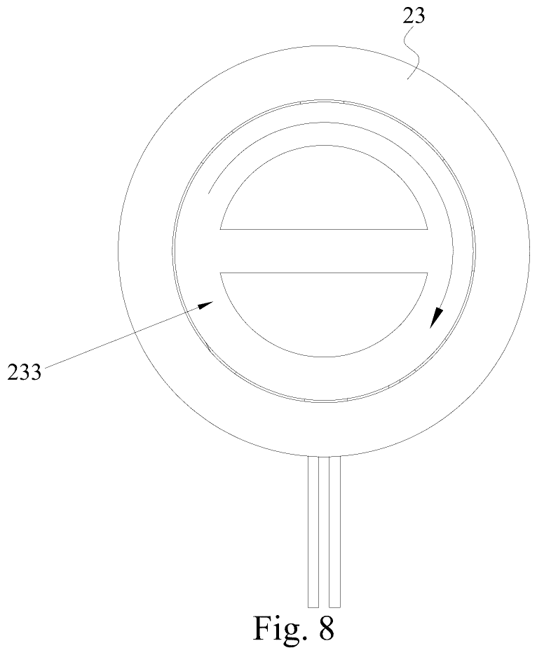

In addition, in order to control the direct conduction or indirect convection by the flexible bag 3 and the coil assembly 9 for cooling the pushing portion 233 has a ring edge 2330 and a pushing member 2331 embedded in the ring edge 2330 and pivoted with the ring edge 2330, such that when the pushing member 2331 rotates clockwise or counterclockwise due to an external force, the pushing member 2331 moves close to or away from the front cover 22 to change the size of the accommodating space 20, so as to move all or part of the flexible bag 3 accordingly to adjust the cooling performance of the flexible bag 3 on the coil assembly 9. The flexible bag 3 is in direct contact with the coil assembly 9; or close to the coil assembly 9 with a gap smaller than 1 cm in order to achieve a better heat dissipation effect. During the installation process, the pushing member 2331 is rotated counterclockwise and withdrawn as shown in FIG. 7, the flexible bag 3 is placed on a side of the rear cover 23 as shown in FIG. 5, the front cover 22 is magnetically attached to the rear cover 23 by a magnet as shown in FIG. 6. In FIG. 8, the pushing member 2331 rotates clockwise to push forward until resistance is felt. At this time, the flexible bag 3 attaches closely to the coil assembly 9 and absorbs heat generated by the coil assembly 9. For disassembly, the front cover 22 and the rear cover 23 are removed, and the flexible bag 3 is removed simultaneously.

Of course, the cooling device can also be plugged in or embedded with a plurality of additional auxiliary sensing mechanisms such as installation prompt, temperature prompt, liquid leakage prompt and other corresponding sensors, transmitter or display units in order to enhance the overall function of the cooling device of the present disclosure. Since the parts can be added through existing technology, there is no need to go into details here.

In summation of the description above, the present disclosure allows the stimulation unit of the electromagnetic stimulation apparatus to quickly replace the cooling source and is suitable for the user to maintain a tolerable temperature when repeatedly performing stimulation for improvement, and for the more expensive heating source coil to be sealed with the cover body as a single unit, thus preventing the device, unit and apparatus from being damaged by collisions occurred when separating and replacing the heating source, and the use of the extrusion part to correspondingly move the flexible bag body in whole or in part and thus adjust the cooling effect of the flexible bag body on the coil group. The cooling effect of the flexible bag body on the coil assembly can be adjusted by the pushing part to move all or part of the flexible bag accordingly. By using the heat-conducting principle of the coil assembly, which is a metal, and the heat-absorbing principle of the cooling liquid, the temperature of the coil assembly is transferred to the cooling liquid in the flexible bag. Since the specific heat of the cooling liquid is much greater than that of the coil assembly, it can store the heat energy of the coil assembly and slow down the temperature rise. The cooling method of the present disclosure, though seemingly simple, solves a number of practical problems, including: (1) It features low cost and small volume. (2) Even without replacing the flexible bag, the temperature of coil assembly can be maintained within a range acceptable to human body for more than 30 minutes. (3) There is no immersion liquid issue. (4) There is no noise issue. (5) Since the flexible bag is installed on the back side of the coil assembly, it does not reduce the strength or affect the stimulation. (6) It features simple disassembly, no wiring, and easy use by the general public. (7) The flexible bag can be removed for a low-intensity or short-term use to achieve light weight. In short, the present disclosure is able to maintain the low temperature operation of the coil assembly and at the same time has the advantages of low volume, consistent efficiency, easy-to-maintain, no noise, low cost, low weight, etc.

1. A cooling device for a stimulation unit of an electromagnetic stimulation apparatus, the stimulation unit comprising a coil assembly, and the cooling device comprising:
a casing having an accommodating space defined therein and an opening formed thereon, the accommodating space being provided for accommodating the coil assembly, the opening being provided for passing and installing a plurality of power wires electrically coupled to the coil assembly, and the casing comprising:
a front cover being a disk structure, and the coil assembly being fixed to a side of the front cover, wherein a periphery of the front cover is provided with a plurality of first fixed parts, and the front cover and the coil assembly are fixed as a whole by an encapsulant; and
a rear cover being a cover structure and having a bottom and a circular lateral portion, wherein an edge of the circular lateral portion is provided with a plurality of second fixed parts opposite the plurality of first fixed parts, the plurality of first fixed parts is fixed and configured to be corresponsive to the plurality of second fixed parts, and the accommodating space is defined between the front cover and the rear cover; and
a flexible bag installed in the accommodating space and filled with a coolant,
wherein, after the rear cover and the front cover are installed, the flexible bag provides a cooling effect to the coil assembly through convection or conduction.

2. The cooling device according to claim 1, wherein the rear cover and the flexible bag are fixed as a whole.

3. The cooling device according to claim 2, wherein the first fixed part and the second fixed part are magnetic attachment structures.

4. The cooling device according to claim 1, wherein a bottom of the rear cover is provided with a pushing portion, the pushing portion has a ring edge and a pushing member embedded in the ring edge and pivoted with the ring edge, and when the pushing member rotates clockwise or counterclockwise under an external force, the pushing member moves close to or away from the front cover to change a size of the accommodating space, so as to move all or part of the flexible bag accordingly to adjust the cooling effect of the flexible bag on the coil assembly.

5. The cooling device according to claim 4, wherein the first fixed part and the second fixed part are magnetic attachment structures.

6. The cooling device according to claim 5, wherein the coolant is water or a non-metallic liquid with a magnetic permeability smaller than 1*10\hat{ }−5 Henrys/meter (H/m) and with a specific heat capacity greater than 1,000 Joules/(kg*K).

7. The cooling device according to claim 6, wherein the flexible bag is directly in contact with the coil assembly, or
the flexible bag is indirectly in contact with the coil assembly with a gap smaller than 1 cm.

8. The cooling device according to claim 1, wherein the first fixed part and the second fixed part are magnetic attachment structures.

9. The cooling device according to claim 8, wherein the coolant is water or a non-metallic liquid with a magnetic permeability smaller than 1*10\hat{ }−5 Henrys/meter (H/m) and with a specific heat capacity greater than 1,000 Joules/(kg*K).

10. The cooling device according to claim 9, wherein the flexible bag is directly in contact with the coil assembly, or
the flexible bag is indirectly in contact with the coil assembly with a gap smaller than 1 cm.

11. The cooling device according to claim 3, wherein the coolant is water or a non-metallic liquid with a magnetic permeability smaller than 1*10\hat{ }−5 Henrys/meter (H/m) and with a specific heat capacity greater than 1,000 Joules/(kg*K).

12. The cooling device according to claim 11, wherein the flexible bag is directly in contact with the coil assembly, or
the flexible bag is indirectly in contact with the coil assembly with a gap smaller than 1 cm.
